(19) United States
(12) Patent Application Publication
(10) Pub. No.: US 2022/0313675 A1
(43) Pub. Date: Oct. 6, 2022

(54) Anti-Aging Telomere-Strengthening NAD+ Sirtuin Dietary Supplement Composition

(71) Applicant: Larry Shultz, Beverly Hills, CA (US)

(72) Inventor: Larry Shultz, Beverly Hills, CA (US)

(21) Appl. No.: 17/713,204

(22) Filed: Apr. 4, 2022

Related U.S. Application Data
(60) Provisional application No. 63/170,651, filed on Apr. 5, 2021.

(51) Int. Cl. A61K 31/455; A61K 31/405; A61K 31/05; A61K 31/353; A61K 31/352; A61K 31/19; A61K 9/00; A23L 33/00; A23L 33/175; A23L 33/15; A23L 33/105; A23L 2/60

(52) U.S. Cl. CPC A61K 31/455; A61K 31/405; A61K 31/05; A61K 31/353; A61K 31/352; A61K 31/19; A61K 9/0053; A23L 33/40; A23L 33/175; A23L 33/15; A23L 33/105; A23L 2/60; A23V 2002/00

(57) ABSTRACT

The present invention relates to an all-in-one anti-aging composition for a Chromosomal Telomere-Strengthening NAD⁺ Sirtuin Dietary Supplement for concurrent AMPK-activation and CD38-Inhibition comprising: NAD precursor; at least one polyphenol capable of providing simultaneous AMPK-activation and CD38 inhibition to increase activation of the seven Sirtuins; at least one Telomerase-activator to increase chromosomal telomere length, and C8 Caprylic Acid MCT ketones that increase NAD+ and cellular immunometabolic effectiveness.

### TECHNICAL FIELD

The present invention generally relates to a dietary supplement composition for providing a user with an anti-aging chromosomal telomere-strengthening NAD⁺-sirtuin dietary supplement for concurrent AMPK-activation and CD38-inhibition to replenish and increase the intracellular nutrients needed by the human body to restore cellular homeostasis and delay, or even reverse, progression of the mitochondrial, immunological and metabolic disorders, neurodegenerative diseases and chronic inflammatory diseases associated with human aging.

### BACKGROUND OF THE INVENTION

Aging is a fundamental process of life. While people aged 65 and older are considered elderly senior citizens, every young and middle-age adult will experience a gradual but continuous decline in their physical and mental abilities as they grow older. that Old age brings new wrinkles, gray hair and loss of muscle mass and memory because the aging body's metabolism, mitochondrial ATP chemical-energy production and immune response systems slow down, producing declining amounts of the vital enzymes, amino-acids and proteins that a healthy body needs to keep living cells functioning at optimal levels in homeostatic balance. Instead, these chemical shortages in the aging body cause life-threatening genomic instability, telomere attrition, epigenetic alteration, loss of proteostasis, deregulated nutrient-sensing, mitochondrial dysfunction, cellular senescence, stem cell exhaustion, altered intercellular communication, telomere-weakening and metabolism unbalancing. Moreover, some degree of cognitive brain impairment, including decline in cognitive function that worsens with age, and age-related changes in brain morphology and cerebrovascular function, are commonly observed in aging brains. Cognitive decline has been consistently reported with aging across a range of cognitive domains, including processing speed, attention, episodic memory, spatial ability and executive function.

In addition to direct cellular damage, the brain is also indirectly impaired by injuries to micro-vascular structures. It is evident that the pathology of aging and also dementia involves a complexity of these interacting factors, which are linked together. For example, mitochondrial dysfunction leads to less cellular ATP chemical energy being produced, which shortage has cascading negative effects on bodily health, including increased oxidative stress that can trigger inflammation, vascular insults and cerebrovascular micro-strokes to the brain. The elderly human body is susceptible to such degradation because the aging body no longer produces enough of its own mitochondrial energy and chemical-substrates to launch an adequate immune response to protect itself from bacterial and viral attack. By supplementing these vital nutrients and precursors lost to age, an elderly body can more effectively fight illnesses and diseases by stimulating its metabolism and increasing its mitochondrial energy and immune response to ever-changing environmental stresses and contaminants.

Scientific and medical research studies have separately confirmed that the enzyme, Nicotinamide adenine dinucleotide (NAD⁺), is present at about three grams in the average person, plays key roles in the regulation of almost all major biological processes, and is required for approximately 500 different enzymatic reactions in the human body. NAD+ works as a substrate for three important biochemical reactions, including ADP-ribosylation of proteins (catalyzed by PARPs for DNA repair), formation of cyclic ADP-ribose (catalyzed by CD38 in the calcium signaling cycle for skeletal muscle contractions) and histone/protein deacetylation by the Sirtuin family of proteins. NAD⁺ depletion has been associated with hallmarks of aging and underlie a wide-range of age-related diseases, such as metabolic disorders, cancer and neurodegenerative diseases. Emerging evidence confirms that elevation of NAD⁺ levels may slow or even reverse the aspects of aging, and also delay the progression of age-related diseases to bodily organs.

NAD⁺ levels decline with aging. By the time we reach middle age, levels of NAD⁺ can be about half of youthful levels, and fall even further in older age. This is why using nutraceutical dietary supplements to boost NAD⁺ levels (and other specific nutrients and chemical compounds) in older people may emerge as a viable therapeutic strategy to counter aging-associated pathologies and/or accelerated aging. CD38 is a major consumer of NAD⁺ in mammals. Expression and activity of CD38 actually increases over time, with NAD+-consuming CD38 levels being several fold higher in some tissues and cells of the elderly. Since the three NAD⁺-consuming pathways—sirtuins, PARPs, and CD38—compete for the same finite pool of NAD⁺, when too many CD38 molecules bind with and breakdown too many NAD+, there is less NAD+ available in the body to stimulate sirtuins and PARPs. The way to reverse declining levels of NAD+ is to reduce availability of CD38 to bind with NAD+ by providing CD38-inhibiting polyphenols to bind with CD38 instead. This indirectly boosts NAD⁺, leaving more of it available to be used by sirtuins and PARPs. NAD-consuming CD38 protein levels increase in various tissues during aging with a concurrent drop in NAD⁺ levels, causing defective mitochondria, decreased oxygen consumption and increased lactate production (Barbosa et al., 2007; Camacho-Pereira et al., 2016). To conserve NAD+ in the body for upregulating Sirtuin proteins to bind with NAD+, instead of NAD-consuming CD38 proteins and DNA-repairing PARPs competing for available NAD+, there is a need for a dietary supplement that inhibits bonding of CD38 with NAD+ in favor of the more beneficial use of NAD+ to stimulate the body's production of Sirtuins. Sirtuins (SIRTs) are NAD⁺-dependent protein deacetylases, lysine malonyl transferases or ADP (adenosine diphosphate) ribosyl transferases that remove acetyl groups from a wide range of proteins that have been demonstrated to be critical in longevity pathways and control of metabolic enzymes. Sirtuins use and degrade NAD⁺ as part of their enzymatic reaction, which makes NAD⁺ a limiting factor for sirtuin activity.

The ability of NAD⁺ to affect metabolic diseases is mediated by sirtuins. Seven different Sirtuins (SIRT1-7) directly influence all human cellular processes, including DNA/RNA transcription, cellular apoptosis, senescence, immune response inflammation, stress resistance and mitrochondrial chemical energy efficiency, all of which regulate human metabolism, mitrochondrial energy production, stress responses and the aging process itself. AMP-activated protein kinase (AMPK) and the histone/protein deacetylase Sirtuins are fuel-sensing molecules that coexist in human cells. AMPK and Sirtuins both regulate each other and share many common target molecules. The best way to increase cellular expression of Sirtuins to activate AMPK to provide the ATP chemical energy needed to target Sirtuin performance. AMPK is a Master Metabolic Regulator. When a cell's energy state is diminished, AMPK activation restores energy balance by stimulating catabolic processes that generate ATP and downregulating anabolic processes that consume ATP, but are not acutely needed for survival. AMPK and SIRT mediate the biological response of cells to nutrient availability, and in doing so they regulate, often interdependently, metabolism, gene expression, and many important aspects of cell biology. The resultant changes in enzymes, transcriptional activators and coactivators, and the genes they regulate, act predictably to maintain cellular function and integrity. Recent studies have revealed that various plant-based Polyphenols (flavonoids, isoflavonoids, stilbenes, catechins, anthocyanins, proanthocsyanidins, isoflavones and tannins), are natural activators of AMP kinase that work by activating the Sirtuin gene transcription factor that, in turn, activates AMPK, which controls the body's metabolism, including the repair of damaged tissues. Polyphenols are the complex chemicals that give plants their color. The more colorful the plant, usually the higher the polyphenol content. This is why vegetables and fruits are the plants that have the highest concentrations of polyphenols. There is a great deal of epidemiological data that shows the increased dietary intake of foods rich in polyphenols (primarily vegetables and fruits) are associated with lower rates of chronic disease and mortality. We also know that increased levels of polyphenols in the urine (indicating their absorption into the blood) are also strongly associated with reduced mortality and frailty in elderly populations. A 25-55% decrease in the NAD⁺/NADH ratio in the brain has been reported in elderly (frontal and occipital lobes) and in psychiatric disorders, such as schizophrenia and bipolar disorder (frontal lobes), together with a reduction in total NAD. Research has found that high Ketones produce more NAD+ in the brain and reduces inflammation. One study shows that ketones can increase the brain NAD⁺/NADH ratio by about 18% in humans, at least transiently. Therefore, a nutritional ketone intervention, beyond providing an alternative source of energy to the brain, offers the potential to boost the NAD⁺/NADH redox state and may provide additional benefits to the brain such as protection from oxidative stress and inflammation by ketones feeding brain cells instead of glucose. While the brain primarily relies on blood glucose as its predominant energy source, ketones are consumed by the brain as a main source of energy when glucose is limited. Under healthy homeostatic conditions, an increase in brain ketones proportionally decreases brain glucose utilization. Ketone bodies have been shown to reduce ROS (reactive oxygen species) by using NADH as an electron donor, thus increasing the NAD⁺/NADH ratio. Interestingly, while brain glucose utilization decreases in mild cognitively impaired elderly and in Alzheimer's disease patients, ketone metabolism remains intact. Although glucose provides a higher final yield of ATP, the consumption of NAD⁺ is significantly higher in this pathway (4:1). Glucose will reduce 111 molecules of NAD⁺ per 1000 molecules of ATP made, while ketone bodies reduce only 41 to produce a comparable amount of ATP. Decreased use of NAD⁺ by ketone bodies in energy production pathways could increase the amount of free NAD⁺ available as substrate for enzymes and cellular signaling processes. This is why interventions using ketones, or their precursors, have shown therapeutic potential in several neurometabolic disorders, demonstrating the possible value of ketones as an alternative source of brain energy than glucose. Ketones from medium chain triglycerides (MCT) compensate for the brain glucose deficit in Alzheimer's in direct proportion to the level of plasma ketones. While ketones are naturally produced by the body at times of bodily stress from hunger and caloric restriction, most people are unwilling to voluntarily reduce their caloric intake by an amount that is likely to influence their aging while maintaining adequate nutrition. Thus, there has been an increasing focus on developing pharmacological and nutraceutical supplements that can replicate the beneficial effects of caloric restriction on longevity without the need for changing dietary intake.

Apigenin (4,5,7-trihydroxyflavone) is a flavonoid present in vegetables (parsley, celery, and onions), fruits (oranges), herbs (chamomile, thyme, oregano, and basil), and plant-based beverages (tea, beer, and wine). A previous study has shown that apigenin, and other polyphenols, inhibits CD38 from bonding with NAD+, thus increasing NAD⁺ availability. Because levels of NAD-consuming CD38 increase over time in aging bodies, while levels of NAD+ decrease, the aging body has less energy because its cells produce less ATP energy. In addition, as we age, the number of mitochondria per cell is decreasing and so is the protein unfolding process, preventing needed electro-chemical reactions from occurring. The majority of adult human cells also lose the ability to produce enough chromosomal telomerase enzymes in the aging human body to prevent progressive DNA telomere shortening with age, which ultimately leads to the deteriorating effects of aging, leading to death because vital cellular DNA/RNA replication functions are prevented due to weak telomere protection. Telomeres are ends of chromosomes bound by telomere-binding proteins to protect them from being recognized by the body's immune system at the naturally-occurring double-stranded DNA breaks (DSBs) that would otherwise signal the cellular immune response system for disposal. Over time, the shortened telomeres cannot be bound by telomere proteins and are thus exposed as natural DSBs, which activate DNA damage responses and induce RB- and p53-dependent cell cycle arrest. Long telomeres have been associated with longevity in humans, while short telomeres have been associated with cancers, idiopathic pulmonary fibrosis and a variety of proliferative tissue disorders. Environmental factors, including diet and lifestyle factors, affect the rate of telomere shortening which can be reversed by telomerase, the enzyme responsible for adding DNA to the ends of chromosomes, thus maintaining chromosome length. Telomerase activation by natural molecules has been suggested to be an anti-aging modulator that can play a role in the treatment of aging-related diseases by stimulating expression of specific Sirtuins to affect specific processes in the human body to maintain homeostasis. In a recent study, it was found that the administration of nutraceutical supplements may be linked to sustaining the telomere length in healthy adults and reversing the shortening of telomeres by use of the enzyme telomerase. Telomerase-activators have been further demonstrated to enhance the efficiency of the DNA repair process and protect cells from stress and DNA-damaging conditions. In light of the foregoing, there is great need for novel nutraceutical compositions and methods for revitalizing immunologic, metabolic and mitochondrial functions in aging tissues. Such compositions and methods would be useful for the treatment of age-related and mitochondrial diseases, as well as for increasing stress resistance, improving resistance to hypoxia and extending the lifespan of organisms and cells.

As growth of the older “senior citizen” population outpaces total population due to increased lifespans and improvements in medical knowledge and treatment technologies, the proportion of older persons relative to the rest of the population continues to increase considerably. While one in every twelve individuals was at least 60 years of age in 1950, and one in every ten was aged 60 years or older by the end of 2000, the number of over-60 year olds is projected to be one in every five, almost double, by 2050. Hence, with the increase in the older population it has become a necessity to protect the health of the world's aging, elderly baby boomer populations by providing the latest herbal supplements based on the latest scientific research that can replenish all the key NAD+, AMPK-activating enzymes and SIRT proteins that aging bodies stop producing in sufficient amounts to maintain the optimal ratios of water and electrolyte balance, blood pressure, body temperature, respiration, extracellular fluid pH, concentrations of sodium, potassium and calcium ions, glucose sugar and other enzymes and proteins vital for maintaining homeostasis in the human body in response to any change in the environment, diet or level of physical activity.

To date, no efficient non-prescription herbal remedy, nor methods of therapy, can thoroughly revitalize elderly metabolism by ingestion of an all-in-one dietary supplement to replenish the aging body's reduced production of NAD+, AMPK-activators and Sirutin while inhibiting destruction of NAD+ by NAD-consuming CD38 enzymes.

A number of solutions have been introduced in order to solve the abovementioned problems. In one of the closest relevant arts, US20170290768 A1 discloses a composition-of-matter. The present composition includes dietary supplements capable of reducing or reversing the negative effects of alcohol on motor and cognition, dietary supplements having anti-gastroparesis, antiemetic, analgesic and anti-inflammatory activities and/or dietary supplements capable of increasing alcohol catabolism and decreasing the level of toxic products of alcohol catabolism.

Another relevant art WO2019242983 A2 discloses a composition that can treat or prevent at least one physical state selected from the group consisting of oxidative stress, a condition associated with oxidative stress, a reduced level of glutathione, and a condition associated with a reduced level of glutathione, in particular neurodegenerative disease, depression, anxiety, decreased/low motivation, impaired cognitive function, mild cognitive impairment. The composition contains an effective amount of a combination of a Nicotinamide Adenine Dinucleotide (NAD⁺) precursor and at least one ketone or ketone precursor. The composition can be an enterally administered composition such as an orally administered food product or food supplement.

Another relevant art US2019358184 A1 discloses a method of treating an amyloid-3 peptide disease in a subject in need thereof, the method comprising administering to the subject a therapeutically effective amount of a compound that enhances mitochondrial proteostasis.

In Another relevant art US20180071273 A1 discloses nutritional compositions comprising a synergistic effective amount of a NAD-precursor in a combination with an ATP booster. The disclosure provides methods and formulations to enhance natural energy. The disclosure also provides methods and formulations treat mitochondrial energy disorders or diseases.

Another relevant art US2007135376 A1 discloses methods for the reduction of mitochondrial oxidative damage and improved mitochondrial efficiency in an animal by administration of medium chain triglycerides or prodrug of medium chain triglycerides to the animal are provided.

Another relevant art KR20180006389 a discloses methods and compositions for preventing or treating peripheral neuropathy, cognitive deficits, inactivity, depression, chemotherapy and/or radiation therapy induced peripheral neuropathy and cognitive deficits in a subject in need thereof, and improving cognitive performance. The disclosed methods comprise administering to the subject an effective amount of an agent that increases the level of NAD in the subject.

The prior arts described above indicate that studies have been carried out to develop methods and compositions, including dietary supplements, which are capable of reducing or reversing the negative effects of alcohol on motor and cognition. Moreover, the prior arts does not disclose a new kind of method of therapy using this unique combination of NAD-precursors, AMPK-activators, CD38-inhibitors, Telomerase-activators and Ketones all-in-one daily dosage to thoroughly replenish and revitalize elderly metabolism by infusion of these vital supplements to which the latest scientific and medical research confirms to extend the quality of life of elderly people.

The present invention solves the quality of life problem of world's aging elderly baby boomers by providing them with the latest non-prescription herbal supplements that can replenish all the key NAD-precursors, AMPK/Sirtuin/Telomerase-activating and CD38-inhibiting enzymes and proteins needed to improve the healthy function of aging bodies. Moreover, the present invention provides ultimate combinations of AMPK-Activators and Sirtuin-Activators for any given disease/ailment by the Sirtuins affecting such disease/ailment.

The present invention imparts advantages over the existing arts by providing cheaper and safer AMPK-Activating/CD38-Inhibiting NAD⁺ Sirtuin Diet Therapy, and other different ingredient-based supplement programs to revitalize their metabolisms and flush dead senescent cells out of the body, than to have the costly hospital system deal with these patients when they get sick from lack of these life-extending enzymes and proteins.

Hence, there remains a great social need for lower cost, non-prescription nutraceutical herbal supplements that can cure and prevent diseases based on the latest scientific studies for rejuvenating and super-charging the human body's ability to increase synthesis of the aging body's own energizing AMPK-activators, life-extending NAD⁺ enzymes, Telomerase-activators, Ketones and Sirtuin proteins to restore aging metabolisms and immune response systems to an optimal homeostasis.

All the problems, disadvantages and the limitations of the above-mentioned relevant and conventional arts are being overcome by the method and composition of the present invention, which has various technical advancements and certainly economic benefits over the conventional arts.

### SUMMARY OF THE INVENTION

The objective of the present invention is to provide a much needed and highly effective dietary supplement composition for use as an anti-aging AMPK-activating/CD38-inhibiting/telomere-strengthening NAD⁺ sirtuin diet therapy that increases levels of intracellular nicotinamide adenine dinucleotide (NAD⁺) in an amount sufficient to activate selected sirtuins, matched to the human ailments and symptoms they affect. In view of the foregoing disadvantages inherent in the known types of therapeutic telomere-strengthening and metabolism balancing sirtuin diets now present in the prior art, the present invention provides an improved composition of dietary supplements for an AMPK-Activating/CD38-Inhibiting/Telomere-Strengthening NAD⁺ sirtuin diet therapy. As such, the general purpose of the present invention, which will be described subsequently in greater detail, is to provide a new and improved, composition which can be sold to the public under local FDA (Food and Drug Administration) regulations as a qualifying nutraceutical or bioceutical dietary supplement, which do not require a doctor's medical prescription for use or purchase.

The present invention discloses a safe, natural, and environmentally-friendly composition and more specifically, in one embodiment, the present invention is a nutraceutical dietary supplement composition for improved sirtuin diet therapy and sirtuin diet therapy with ketone brain energizer formulation. In another embodiment the composition of four ingredients is provided in a stabilized form.

In one of the embodiments, the present invention mainly pertains to a dietary supplement composition comprising: a) NAD precursor; b) at least one polyphenol capable of providing AMPK-activation; c) at least one polyphenol capable of providing CD38-inhibition; d) at least one Telomerase-activator to increase chromosomal telomere length;

In another important embodiment, the present invention discloses a dietary supplement composition comprising: a) NAD precursor; b) at least one polyphenol capable of providing AMPK-activation; c) at least one polyphenol capable of providing CD38-inhibition; d) at least one Telomerase-activator to increase chromosomal telomere length; and e) C8 Caprylic Acid MCT ketones which by-pass stomach via portal vein for maximum bloodstream absorption;

In another embodiment of the present invention, the main components of said composition comprising the NAD precursor, mainly consist of Niacin/Nicotinic Acid or Nicotinamide versions of Niacin which are known to be converted by the body into NAD+. Meanwhile, the dietary supplement of the present invention also contains either or both of single-function or dual-function polyphenols that can activate AMPK and inhibit CD38 binding of NAD, which polyphenols are selected from Quercetin, Resveratrol, Apigenin, Luteolin, Proanthocyanidins, Kuromanin chloride and Cyanidin. Polyphenol-based AMPK Activators stimulate production of specific Sirtuins to fight disease and maintain cellular homeostasis in different organs of the body. Subsequently, the currently discussed dietary supplement composition additionally comprises C8 Caprylic Acid Ketones, which is helpful in boosting cellular energy and increase in ATP/NAD+, and C8 Caprylic Acid Ketones also replace glucose in the brain to increase cognitive ability lost to aging. The composition further comprises Telomere-increasing Telomerase-activators, which are selected from Oleanolic Acid sources of Bilberry Fruit Extract (Vaccinium Myrtilius) and Olive (Olea Europaea) Skin Extracts, or sources of Gotu Kola (Centella Asiatica) Extract, which increase the Telomerase enzyme, linked to telomere-lengthening, life-extension and cellular homeostasis in the body and brain.

The dietary supplement composition includes NAD-precursors, selected from Nicotinic Acid and Nicotinamide, which are the lowest-cost Niacin substrates that are both highly capable of increasing user's bloodstream NAD⁺ levels to produce more Sirtuins and Telomerase to extend the lifespan and homeostasis of a user's immune and metabolic systems, and also to increase NAD+ substrate levels in aging human blood stream and cells.

The dietary supplement composition can replenish and increase NAD+ levels in aging human blood stream and cells. It is also capable of inhibiting prevalent CD38 NADase from binding to replenished NAD+ substrates. The dietary supplement composition also activates cellular AMPK to produce ATP chemical energy to stimulate production of specific Sirtuins to fight disease and maintain cellular homeostasis, replace glucose in brain with brain-clearing Ketones to increase cognitive ability lost to aging and can activate increase in Telomerase to increase length of chromosomal telomeres linked to life-extension and cellular homeostasis in the body and in the brain.

In another embodiment of the present invention, the dietary supplement composition is provided or administered in the form of pills and/or capsules. Moreover, the composition is also administered in the form of food-grade protein shake powder mix, or as bottled-drink, or in Jello-type fruit Cups or fruit Bars or fruit roll/strips/gummies. Additionally, said dietary supplement composition comprises 1%-5% by weight of a non-glycemic sweetener for food-grade protein shake powder mix, or as bottled-drink, or in Jello-type fruit Cups or fruit Bars or fruit roll/strips/gummies to mask its taste. The said non-glycemic sweetener is selected from Stevia and/or other natural sweetener. Also, the non-glycemic sweetener is optionally added to the composition when the delivery method is selected from one of protein shake powder mix, bottled-drink, Jello-type fruit cups, frozen desserts, fruit-bars or fruit roll/strips/gummies.

In various embodiments of any of the above aspects or any aspect delineated herein, the dietary supplement composition of the invention has the potential to super charge human body production of Telomerase and Sirtuins, and inhibit proliferation of NAD-consuming CD38. The present invention is a new kind of composition using NAD-Precursors, polyphenols as both AMPK-activators and CD-38-inhibitors, telomerase-activators and C8 Caprylic Acid MCT ketones all-in-one dietary supplement capable of revitalizing elderly metabolism by consumption and replenishment of those vital nutrients/enzymes/proteins needed by the aging body for optimal health.

In yet another important technical aspect of the present invention, the composition of the dietary supplement and the method of delivering the dietary supplement using the current methodological improvement have never been previously offered for sale or commercialized all-in-one packaged dosage. This dietary supplement product uses ingredients separately proven capable of enhancing the health and longevity of human body and brain. It has numerous applications such as in the field of health science, nutrition and essential food supplements to activate any body's production of NAD+, AMPK, Telomerase and Sirtuins to heal ailments and restore cellular metabolism to homeostasis, especially in the elderly, who need to replenish vital brain and body nutrients lost to aging.

Other features and advantages of the invention will be apparent from the detailed description, and from the claims. Thus, other aspects of the invention are described in the following disclosure and are within the ambit of the invention.

### DETAILED DESCRIPTION OF THE INVENTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The description used herein are intended merely to facilitate an understanding of ways in which the embodiments herein may be practiced and to further enable those of skill in the art to practice the embodiments herein. Accordingly, the description or explanation should not be construed as limiting the scope of the embodiments herein.

Various modifications to the disclosed embodiments will be readily apparent to those skilled in the art, and the general principles defined herein may be applied to other embodiments and applications without departing from the spirit and scope of the present invention. Thus, the present invention is not limited to the embodiments shown, but is to be accorded the widest scope consistent with the claims. The terms like can be, shall be, could be, and other related terms herein disclosed in the foregoing and later parts of the specification in any means do not limit or alter the scope of the present invention. The terms are provided just for the mere understanding of the main invention and its embodiments.

The present invention relates to an anti-aging telomere-strengthening NAD+-sirtuin dietary supplement composition.

The dietary supplement composition of the preferred embodiment of this invention includes a Nicotinamide adenine dinucleotide (NAD) precursor, at least one polyphenol capable of single or dual-use benefits, providing AMPK-activation and/or CD38 inhibition, with at least one or more polyphenols. None of the prior arts have yet discovered the extreme effectiveness of this unique dietary supplement composition for improving the NAD⁺, AMPK, Sirtuin, Telomerase and Ketone pathways to improved health all-in-one dietary supplement. The present invention provides a new herbal supplement and delivery method to consumers, based on ingredients of a tasty protein shake or gelatin gummy bars or regimen of pills/capsules that the consumer can consume throughout the day based on a daily minimum dosage per day to rejuvenate their NAD⁺ AMPK, Sirtuin, Telomerase and Ketone pathways to health.

In an embodiment of the present invention, the invention relates to a non-prescription herbal remedy combining one or more NAD precursors; at least one polyphenol capable of CD38-inhibition; at least one polyphenol capable of AMPK-activation to determine the Sirtuins to be activated. The present invention combines all legally available herbal extracts to create a multi-ingredient cocktail of activators and inhibitors that work together to more effectively stimulate net NAD+, AMPK, Sirtuin and Telomerase production in the body, thereby inhibiting the destruction of NAD supplements in the body. Furthermore, the present invention provides the precursors to make the NAD and provide the Inhibitors to protect that NAD from being destroyed by prolific CD38 enzymes, while increasing SIRT production from the different AMPK-Activators that activate the seven different SIRTs 1-7 to impact multiple immunometabolic processes and diseases in the human body. The invention pertains to life extension therapy which is focused for elderly people over 60 years old who want to reverse the effects of aging by replenishing vital nutrients previously lost to aging.

In an embodiment of the present invention, the invention provides a food based health therapy of protein shakes, fruit cups, frozen desserts, gummy bars that contain multiple NAD precursors, at least one polyphenol capable of single or dual-use providing AMPK-activation and/or CD38 inhibition; and at least one more polyphenol, with non-glycemic sweeteners in order to provide the elderly human body renewed mitochondrial energy and chemical processes that can cure disease by eliminating the immunological responses that cause the human body's own immune system to attack its own cells. This is what NAD⁺ and Sirtuin supplementation has been shown in multiple scientific and medical studies to prevent and cure.

Accordingly, in another embodiment of the present invention, the NAD precursor is utilized in order to increase human Sirtuin blood levels and extend the quality of life by upregulation of specific Sirtuins to affect specific ailments, as confirmed by the latest scientific, academic and medical research databases. Nicotinamide adenine dinucleotide (NAD) precursors are directly linked with the aging of the human body, and are an important component of the dietary supplement of the present invention. The present disclosure provides a dietary supplement composition in which the first component of the dietary supplement composition is a NAD precursor. It is further disclosed that the preferred NAD precursor is Niacin/Nicotinic Acid to increase the NAD⁺ levels. NAD research has linked NAD+ levels in reducing hearing loss, obesity, diabetes, kidney disease, heart disease, non-alcohol/alcoholic fatty liver disease, muscular dystrophy, muscular mitochondrial disease, stroke, neurodegeneration-related disease, aging, longevity, healthspan, progeroid syndromes, genetic macular degeneration and cataract.

Furthermore, the second component of the dietary supplement composition comprises a CD38-inhibitor that can be provided by one or more dual-use polyphenols. The at least one polyphenol of the dietary supplement composition that has dual-use as both an AMPK-activator and a CD38-inhibitor is selected from the group of Resveratrol and Apigenin or Quercetin, Luteolin, Proanthocyanidins, Kuromanin chloride or Cyanidin.

In another embodiment of the present invention, the dietary supplement composition is administered to the subject in the form of pills and/or capsules. Furthermore, the dietary supplement composition is administered to the user in the form of protein shake powder mix, or as bottled-drink, or in Jello-type fruit cups, frozen desserts, fruit bars or fruit roll/strips/gummies depending upon the desired form of consumption by the patient/subject/user in need.

In yet another embodiment of the present invention, the dietary supplement composition comprises of 1%-5% by weight of a non-glycemic sweetener for protein shake powder mix, or as bottled-drink, or in Jello-type fruit cups, frozen desserts, or fruit bars or fruit roll/strips/gummies. Further, the non-glycemic sweetener is selected from stevia and/or other natural sweetener, like erythritol and monk-fruit in order to provide sweetness to the dietary supplement composition which makes the composition luscious. Moreover, the non-glycemic sweetener is optionally added to the composition depending upon the delivery method selected from one of protein shake powder mix, bottled-drink, Jello-type fruit cups, frozen desserts, fruit-bars or fruit roll/strips/gummies.

In yet another embodiment of the present invention, the dietary supplement composition is utilized in the anti-aging Chromosomal Telomere-strengthening NAD⁺-Sirtuin diet regimen for AMPK-activation and CD38-Inhibition.

In yet another embodiment of the present invention, the invention is a dietary supplement composition comprising of NAD precursor; C8 Caprylic Acid MCT ketones which by-pass stomach via portal vein for maximum bloodstream absorption and glucose substitution in the brain; at least one polyphenol capable of CD38-inhibition; and at least one AMPK/Sirtuin-activating polyphenol.

In another embodiment of the present invention, C8 Caprylic Acid Ketone boosts cellular energy and increase in ATP/NAD⁺.

As to the manner of usage and operation of the instant invention, same should be apparent from the above disclosure, and accordingly no further discussion relevant to the manner of usage and operation of the instant invention shall be provided.

Therefore, the foregoing is considered illustrative of only the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

The foregoing discussion is illustrative of the invention. However, since many embodiments of the invention can be made without departing from the spirit and scope of the invention, the invention resides wholly in the claims hereinafter appended.

What is claimed is:

1. An anti-aging telomere-strengthening NAD+-sirtuin dietary supplement composition comprising:
a. NAD precursor;
b. at least one polyphenol capable of providing AMPK-activation;
c. at least one polyphenol capable of providing CD38 inhibition; and
d. at least one Telomerase-activator to increase chromosomal telomere length.

2. The dietary supplement composition according to claim 1, wherein said NAD precursor substrate is selected from Niacin/Nicotinic Acid (NA), Nicotinamide (NAM), Nicotinamide Mononucleotide (NMN), Nictoinamide Riboside (NR) or Tryptophan (Trp).

3. The dietary supplement composition according to claim 1, wherein said at least one polyphenol capable of providing AMPK-activation is selected from one or more phytonutrient extracts of flavonoids, flavones, flavonols, phenolic acids, stilbenes, lignans, catechins, anthocyanins, proanthocyanidins, tannins, hydroxycinnamic acids, and isoflavones.

4. The dietary supplement composition according to claim 1, wherein said at least one polyphenol capable of providing CD38-inhibition is selected from one or more phytonutrient extracts of Resveratrol and Apigenin or Quercetin, Luteolin, Proanthocyanidins, Kuromanin chloride or Cyanidin.

5. The dietary supplement composition according to claim 1, wherein said at least one Telomerase-activator is selected from Oleanolic Acid sources of low-cost Bilberry Fruit Extract (Vaccinium Myrtilius), Olive (Olea Europaea) Skin Extracts, or sources of Gotu Kola (Centella Asiatica) Extract.

6. The dietary supplement composition according to claim 1, wherein said NAD-precursor substrate increases user's NAD⁺ levels in the bloodstream.

7. The dietary supplement composition according to claim 1, wherein said composition is administered in the form of pills, tablets, softgels and/or capsules that may contain an enteric coating or other type polymer barrier to prevent release of the ingredients in the user's stomach before reaching the small intestine, for greater absorption of the ingredients into the bloodstream.

8. The dietary supplement composition according to claim 1, wherein said composition is administered in the form of food-grade protein shake powder mix, or as bottled-drink, or in Jello-type fruit Cups, frozen desserts, fruit Bars or fruit roll/strips.

9. The dietary supplement composition according to claim 8, wherein said composition comprises 1%-5% by weight of a non-glycemic sweetener for protein shake powder mix, or as bottled-drink, or in Jello-type fruit Cups, frozen desserts or fruit Bars or fruit roll/strips.

10. The dietary supplement composition according to claim 1, wherein said composition is used in an anti-aging Chromosomal Telomere-Strengthening NAD⁺-Sirtuin Dietary Supplement for concurrent AMPK-activation and CD38-Inhibition.

11. An anti-aging telomere-strengthening NAD+-sirtuin dietary supplement composition comprising:
a. NAD precursor;
b. at least one polyphenol capable of providing AMPK-activation;
c. at least one polyphenol capable of providing CD38-inhibition;
d. at least one Telomerase-activator to increase chromosomal telomere length; and
e. C8 Caprylic Acid MCT ketones to by-pass stomach via portal vein for maximum bloodstream absorption.

12. The dietary supplement composition according to claim 11, wherein said NAD precursor is Niacin/Nicotinic Acid (NA), Nicotinamide (NAM), Nicotinamide Mononucleotide (NMN), Nictoinamide Riboside (NR) or Tryptophan (Trp).

13. The dietary supplement composition according to claim 11, wherein said at least one polyphenol capable of providing AMPK-activation is selected from one or more phytonutrient extracts of flavonoids, flavones, flavonols, phenolic acids, stilbenes, lignans, catechins, anthocyanins, proanthocyanidins, tannins, hydroxycinnamic acids, and isoflavones.

14. The dietary supplement composition according to claim 11, wherein said at least one polyphenol capable of providing CD38-inhibition is selected from one or more of the phytonutrient extracts of Resveratrol and Apigenin or Quercetin, Luteolin, Proanthocyanidins, Kuromanin chloride or Cyanidin.

15. The dietary supplement composition according to claim 11, wherein said at least one Telomerase-activator is selected from Oleanolic Acid sources of low-cost Bilberry Fruit Extract (Vaccinium Myrtilius), Olive (Olea Europaea) Skin Extracts, or sources of Gotu Kola (Centella Asiatica) Extract.

16. The dietary supplement composition according to claim 11 wherein both said NAD-precursor and C8 Caprylic Acid MCT separately increase NAD⁺ levels.

17. The dietary supplement composition according to claim 11, wherein said composition is administered in the form of pills, tablets, softgels and/or capsules that may contain an enteric coating or other type polymer barrier to prevent release of the ingredients in the stomach before reaching the small intestine, for greater absorption into the bloodstream.

18. The dietary supplement composition according to claim 11, wherein said composition is administered in the form of food-grade protein shake powder mix, or as bottled-drink, or in Jello-type fruit Cups, frozen desserts, fruit Bars or fruit roll/strips.

19. The dietary supplement composition according to claim 11, wherein said composition comprises 1%-5% by weight of a non-glycemic sweetener for food-grade protein shake powder mix, or as bottled-drink, or in Jello-type fruit Cups, frozen desserts, fruit Bars or fruit roll/strips.

20. The dietary supplement composition according to claim 11, wherein said composition is used in a Chromosomal Telomere-Strengthening NAD⁺-Sirtuin Dietary Supplement for concurrent AMPK-activation and CD38-Inhibition.

21. The dietary supplement composition according to claim 11, wherein said C8 Caprylic Acid MCT Ketone replaces glucose for energy in the brain while increasing NAD+ and cellular ATP energy in the body.
